(19) United States
(12) Patent Application Publication
(10) Pub. No.: US 2008/0097333 A1
(43) Pub. Date: Apr. 24, 2008

(54) BEDSIDE ENTANGLEMENT PREVENTION AND TUBING APPARATUS

(76) Inventor: Lenore Therese Henning, Glen Ellyn, IL (US)

Correspondence Address: LENORE T. HENNING, 22 W 549 SECOND PLACE, GLEN ELLYN, IL, 60137, US

(21) Appl. No.: 11/428,329

(22) Filed: Jun. 30, 2006

(51) Int. Cl. A61M 25/02

(52) U.S. Cl. 604/174

(57) ABSTRACT

An apparatus for directing medical tubing away from mechanical portions of a hospital bed and off an underlying floor so as to facilitate identification and prevent entanglement, dislodgement, and contamination of the lines includes a tubular foam clasp for snug-fitting, horizontal attachment to a hospital bedrail having a barrel and on the underside of the body, a longitudinal slit. A series of slots in the body of the clasp extend toward the base flexible polyethylene foam material that can be repeatedly separated without fracturing to facilitating mounting on a hospital bedrail. The apparatus permits transverse insertion of medical tubing into a slot and positioning in a confining aperture, thereby allowing longitudinal and limited lateral movement of the tubing extending from a patient to medical devices involved in the care of patients while preventing entanglement with adjacent medical tubing and mechanical parts of a hospital bed.

### SPECIFICATIONS

The clasp features a one piece, fabricated, cross-linked polyethylene foam tube, which can be repeatedly separated at the longitudinal slit and placed on a hospital bedrail without fracturing. The cylinder is flexible as it is made from cross-linked polyethylene foam. However, the cylinder can be constructed from other materials that can be repeatedly bent without fracturing. Examples of such materials include, but are not limited to, polypropylene, vinyl, nylon, rubber, and leathers.

### Description of the Prior Art

The use of tubing clamps is known in the prior art particularly for use in industrial and automotive applications. In these, a plurality of wires, hoses, tubes, and the like are routed from one location to another. These devices are usually permanently installed and are molded or formed to the diameter of the cylindrical device they are meant to restrain or position. U.S. Pat. No. 3,461,876 to Miller discloses a tubing clamp. U.S. Pat. No. 3,893,468 to McPhee discloses a clamp for flexible tube and method of regulating flow in such a tube. U.S. Pat. No. 4,387,617 to Burns discloses a clasp. U.S. Pat. No. 4,589,171 to McGill discloses a device for holding and positioning tubing of I.V. administration set. U.S. Pat. No. 5,115,542 to Gehres discloses a hose separator clip. U.S. Pat. No. 5,334,186 discloses a device for organizing medical lines that service a patient in a hospital bed U.S. Pat. No 5,336,179 discloses a device for organizing medical lines that service a patient in a hospital bed U.S. Pat. No. 5,389,082 to Bauges et al. discloses an intravenous line separator system. U.S. Pat. No. 5,427,338 discloses a device for organizing medical lines that service a patient in a hospital bed U.S. Pat. No. 5,624,403 discloses a device for organizing medical lines that service a patient in a hospital bed U.S. Pat. No. 5,876,371 discloses a device for organizing medical lines that service a patient in a hospital bed

While these devices fulfill their respective, particular objectives and requirements, the aforementioned patents do not disclose a medical tubing organization system to direct flexible tubes and cords away from mechanical portions of a hospital bed.

Modern hospital beds provide a patient with four bedrails that can be raised independently for ingress and egress. The top two bedrails contain the bed controls. However, when the head of the bed is raised, any medical equipment tubing that is placed across the rail slips downward into the crease of the bed. Medical tubing is abundant in most areas of clinical nursing practice.

Common examples include intravenous (IV) tubing, central lines, oxygen tubing, drains, epidural Analgesia and more. It is not uncommon to see IV lines and other cables tangled when patients are very mobile or in transport. Tubing represents a potential source of complication when it becomes dislodged, impinged, entangled or wrapped around the patient's neck or limbs.

Additionally, the “spaghetti syndrome” of entangled tubing possibly increases the risk of medication error (Lovell et al 2001). Presently there is no commercial product available to prevent the medical equipment tubing from becoming caught in the hinges when the tubing slides down the rail.

### BACKGROUND OF THE INVENTION

1. Field of the Invention

The patient's bedside tubing entanglement prevention clasp is an invention for use in the medical field, and relates to medical equipment tubing, and more particularly to directing the tubings and cords away from mechanical portions of the hospital bed and off the floor.

2. Background Information

This clasp fits onto the upper portion of modem hospital beds, which have four independent siderails that raise and lower to provide easy patient ingress and egress. The clasp is designed for single patient use, and is latex-free, quiet, soft, and safe. Additionally, the mobility of the clasp allows for variable placement on either side of the patient's bed. Certain safety and health problems are avoided when the clasp prevents undo patient harm or accidental patient restraint. Accordingly, the clasp addresses patient safety and patient satisfaction by keeping oxygen tubing, epidural analgesia tubing, naso-gastric tubing, intravenous tubing, and other various lines separated. It prevents tubing from sliding down the railing and directing it away from mechanical portions of the bed when the head of the hospital bed is raised. It eliminates the pain to the patient from the accidental tension on tubing that is connected to him.

Moreover, greater caregiver safety results when patients are provided with access to safety features. It offers patients ready access to a clasp that will allow him to participate in self-care and gain knowledge of safety alternatives. A feature of the invention is the fact that the clasp may be used with many different types of rails and poles. Excellent portability allows for transfer to an IV pole or a wheelchair for use with patient ambulation and transportation. Use of the clasp keeps tubing free from the contamination of being dragged on the floor, being stepped on by the caregiver or by the patient himself, or being caught in the various equipment wheels. The clasp keeps patient's medical equipment tubing aligned, untangled, and free from impingement in the hospital bedrail hinges.

In my experience as a nurse, I have observed that caregivers have improved efficiency when equipment is positioned correctly. However, when the head of the bed is elevated, the medical equipment tubing, which is connected to the patient, is in jeopardy. Patients receiving oxygen therapy usually have the head of the bed elevated to facilitate easier breathing. As patients get in and out of bed the clasp will afford a convenient and clean place to suspend lightweight nasal canula oxygen tubing. Doctors often write orders for “head of bed elevated 30 degrees” while patients receive epidural analgesics. This clasp keeps the epidural tubing aligned near the head of the patient's bed and aids in the prevention of accidental dislodging of the epidural catheter.

Pre and post-operative patients with naso-gastric tubes have a doctor's order for “head of the bed elevated” and “naso-gastric tube to wall suction”. This clasp will keep the tubing near the patient's head and prevent accidental displacement of the naso-gastric tubing. Critical patients in intensive care units often have as many as three intravenous tubings connected to vital central venous catheters and triple infusion pumps. This clasp maintains therapeutic positioning of all lines. Other objects and advantages will become apparent from the specifications and drawings.

### SUMMARY OF THE INVENTION

In view of the foregoing disadvantages inherent in the known types of tubing clamps now present in the prior art, the present invention provides a new patient safety device diverting medical tubing between sources of equipment and a patient away from mechanical portions of the hospital bed and off of the floor.

This easy to use, moveable, soft and safe clasp allows for variable placement on either side of hospital bedrails. Certain safety and health problems are avoided when the clasp prevents undo patient harm or accidental patient restraint. The patient's tubes are placed in the circular spaces through the beveled perpendicular slots on this clasp. The use of this clasp keeps patient's tubing aligned on the bedrail. Additionally, the tubing is free from impingement in the hinges of the bed. Proper placement on the bedrail prevents tubing from dragging on contaminated floors, being stepped on, or being caught under various equipment wheels. Improved patient and caregiver safety and satisfaction occurs when solutions are provided for patient care issues.

### BRIEF DESCRIPTION OF THE DRAWING

FIG. 1 is a top and side perspective view of an embodiment of the entanglement prevention apparatus of the present invention with detailing and measurements.

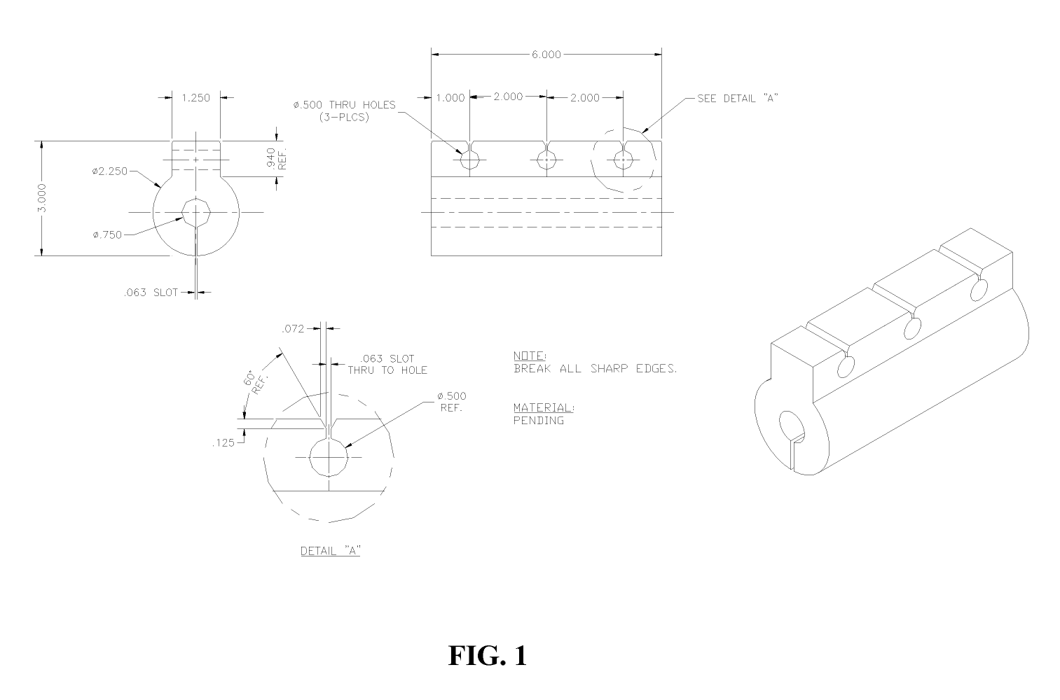

I claim:

1. A device for directing medical tubing away from mechanical portions of a hospital bed and off an underlying floor, said hospital bed having a hinge for raising a head and upper body of a patient and having a bedrail, said bedrail having a hinge for raising and lowering said bedrail with respect to said bed, said device comprising:
a. a sleeve of resilient material with a memory for returning to a predetermined configuration,
b. said sleeve having an inner opening sized to snuggly receive said bedrail,
c. said sleeve having a longitudinal slot forming opposing surfaces, said opposing surfaces separable for the placement of said sleeve around said bedrail, and
d. said sleeve having a plurality of equally sized beveled, circular bores, orientated perpendicularly to longitudinal slot wherein said medical tubing is placed and directed away from said mechanical portions and off said floor.
